(19) United States
(12) Patent Application Publication
(10) Pub. No.: US 2025/0325294 A1
(43) Pub. Date: Oct. 23, 2025

(54) KNIFE DESIGNS FOR END EFFECTORS USED IN SURGICAL TOOLS

(71) Applicant: CILAG GMBH INTERNATIONAL, Zug (CH)

(72) Inventors: Jeff CLARK, Blue Ash, OH (US); James Matthew WILSON, Mason, OH (US); Guowei John ZHANG, Cincinnati, OH (US); Steve SMOLIK, Cincinnati, OH (US)

(73) Assignee: CILAG GMBH INTERNATIONAL, Zug (CH)

(21) Appl. No.: 18/637,604

(22) Filed: Apr. 17, 2024

(51) Int. Cl. A61B 17/295; A61B 17/00; A61B 34/30; A61B 34/37

(52) U.S. Cl. CPC A61B 17/295; A61B 34/37; A61B 2017/00017; A61B 2017/00398; A61B 2017/00477; A61B 2017/00526; A61B 2017/00836; A61B 2017/00867; A61B 2034/302

(57) ABSTRACT

An end effector of a surgical tool includes opposing first and second jaws, a knife slot defined in one or both of the first and second jaws, and a monolithic knife assembly comprising a drive rod and a blade distally extending from the drive rod and extendable through the knife slot, the drive rod and the blade made from a same material.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

Various robotic systems have been developed to assist in MIS procedures. Robotic systems can allow for more instinctive hand movements by maintaining natural eye-hand axis. Robotic systems can also allow for more degrees of freedom in movement by including an articulable “wrist” joint that creates a more natural hand-like articulation. In such systems, an end effector positioned at the distal end of the instrument can be articulated (moved) using a cable driven motion system having one or more drive cables that extend through the wrist joint. A user (e.g., a surgeon) is able to remotely operate the end effector by grasping and manipulating in space one or more controllers that communicate with a tool driver coupled to the surgical instrument. User inputs are processed by a computer system incorporated into the robotic surgical system, and the tool driver responds by actuating the cable driven motion system. Moving the drive cables articulates the end effector to desired angular positions and configurations.

Some end effectors also include a knife that is able to be advanced and retracted between opposing jaws to cut or sever tissue grasped between the opposing jaws. Improvements to the design and function of the knife are desirable to improve the efficiency of the end effector and any procedures undertaken with the end effector, and also to improve the manufacturability of the knife.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIG. 4 is an enlarged isometric view of the distal end of the surgical tool of FIG. 2.

FIG. 5 is another enlarged isometric view of the distal end of the surgical tool of FIG. 2, according to one or more embodiments of the present disclosure.

FIG. 6A is an isometric side view of an example of the monolithic knife assembly utilizable with the surgical tool of FIG. 5, according to one or more embodiments.

FIG. 6B is a side view of the monolithic knife assembly of FIG. 6A.

FIG. 6C is a top view of the monolithic knife assembly of FIGS. 6A and 6B.

FIG. 7 is a front cross-sectional view of the distal end of the surgical tool of FIG. 2, according to one or more embodiments of the present disclosure.

FIG. 8A is an isometric side view of another example of the monolithic knife assembly utilizable with the surgical tool of FIG. 5, according to one or more additional embodiments.

FIG. 8B is a side view of the monolithic knife assembly of FIG. 8A.

FIG. 8C is a top view of the monolithic knife assembly of FIG. 8A.

### DETAILED DESCRIPTION

The present disclosure is related to surgical tools and, more particularly, to end effectors having improved knife attachment and functionality.

Embodiments discussed herein describe an end effector for a surgical tool, where the end effector includes opposing first and second jaws, and a knife slot defined in one or both of the first and second jaws. The surgical tool includes a monolithic knife assembly having a knife blade and a drive rod. The knife blade is extendable through the knife slot and is integrally formed with the drive rod, such that the knife blade and the drive rod are manufactured from the same piece of material. The knife assembly may also include a hinge portion located between a proximal portion of the knife blade and a distal end of the drive rod. In embodiments, the drive rod proximate to the knife blade may have a cross-sectional shape that closely approximates a circle, such as an octagonal shape, such that it exhibits nearly uniform bending modulus in all bending directions. Forming the knife blade and the drive rod from a single piece of material allows for them to be joined without additional components (e.g., ferrules, etc.), which in turn reduces the cross-sectional area of the knife assembly at the hinge portion and the distal portion of the drive rod. Reducing the cross-sectional area of the knife assembly is advantageous as it allows for the knife slot to be correspondingly smaller, which will increase areas of the sealing surfaces of the first and second jaws. Further, forming the knife blade and drive rod from the same piece of material may provide efficiencies during manufacturing, as it will allow the knife blade and the drive rod to be manufactured in a single step and eliminate a step of assembling them together, and moreover permit batch manufacturing.

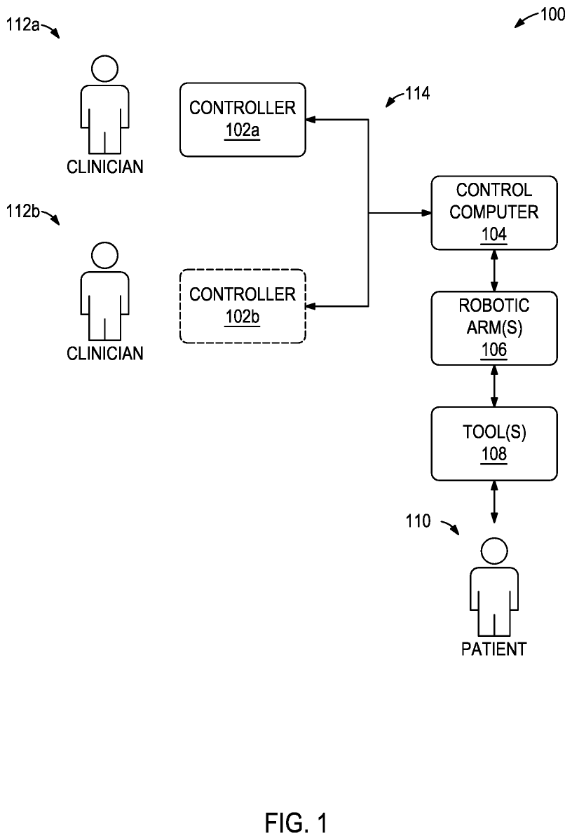

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

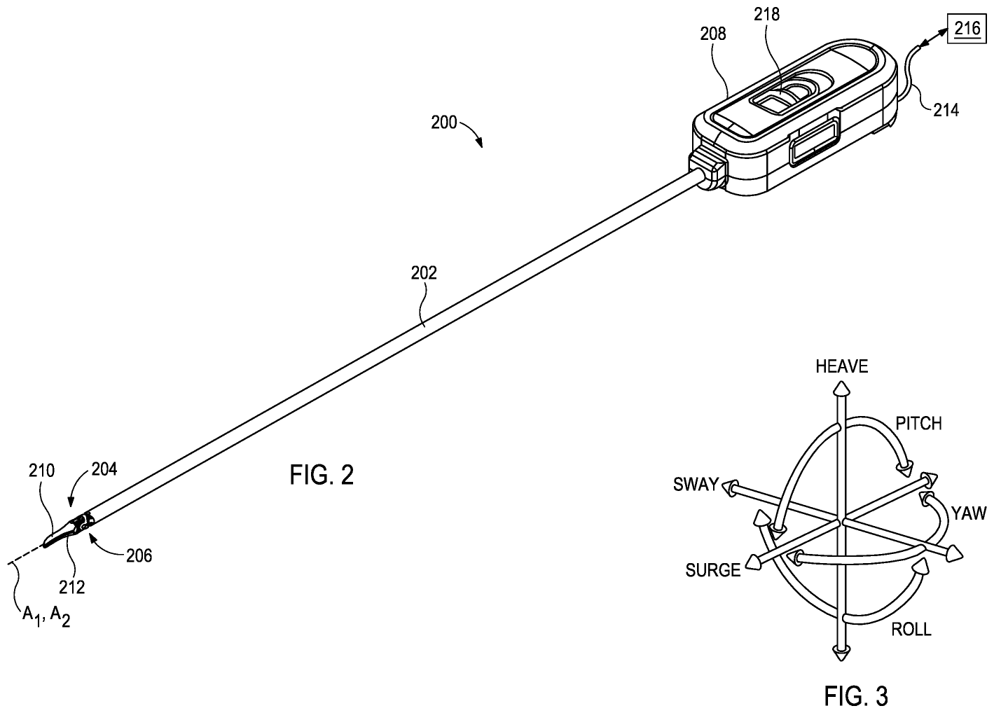

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. Accordingly, the surgical tool 200 may be designed to be releasably coupled to a tool driver included in the robotic surgical system 100. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In applications where the surgical tool is used in conjunction with a robotic surgical system (e.g., the robotic surgical system 100 of FIG. 1), the drive housing 208 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the housing 208 includes (contains) various drive inputs and mechanisms (e.g., gears, actuators, etc.) designed to control operation of various features associated with the end effector 204 (e.g., clamping, firing, cutting, rotation, articulation, etc.). In at least some embodiments, the shaft 202, and hence the end effector 204 coupled thereto, is configured to rotate about a longitudinal axis A₁ of the shaft 202. In such embodiments, at least one of the drive inputs included in the housing 208 is configured to control rotational movement of the shaft 202 about the longitudinal axis A₁.

The shaft 202 is an elongate member extending distally from the housing 208 and has at least one lumen extending therethrough along its axial length. In some embodiments, the shaft 202 may be fixed to the housing 208, but could alternatively be rotatably mounted to the housing 208 to allow the shaft 202 to rotate about the longitudinal axis A₁. In yet other embodiments, the shaft 202 may be releasably coupled to the housing 208, which may allow a single housing 208 to be adaptable to various shafts having different end effectors.

The end effector 204 can exhibit a variety of sizes, shapes, and configurations. In the illustrated embodiment, the end effector 204 comprises a combination tissue grasper and vessel sealer that include opposing first (upper) and second (lower) jaws 210, 212 configured to move (articulate) between open and closed positions. As will be appreciated, however, the opposing jaws 210, 212 may alternatively form part of other types of end effectors such as, but not limited to, surgical scissors, a clip applier, a needle driver, a babcock including a pair of opposed grasping jaws, bipolar jaws (e.g., bipolar Maryland grasper, forceps, a fenestrated grasper, etc.), etc. One or both of the jaws 210, 212 may be configured to pivot to articulate the end effector 204 between the open and closed positions.

FIG. 3 illustrates the potential degrees of freedom in which the wrist 206 may be able to articulate (pivot) and thereby move the end effector 204. The wrist 206 can have any of a variety of configurations. In general, the wrist 206 comprises a joint configured to allow pivoting movement of the end effector 204 relative to the shaft 202. The degrees of freedom of the wrist 206 are represented by three translational variables (i.e., surge, heave, and sway), and by three rotational variables (i.e., Euler angles or roll, pitch, and yaw). The translational and rotational variables describe the position and orientation of the end effector 204 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system configured to facilitate actuation and articulation of the end effector 204 relative to the shaft 202. Moving (actuating) one or more of the drive cables moves the end effector 204 between an unarticulated position and an articulated position. The end effector 204 is depicted in FIG. 2 in the unarticulated position where a longitudinal axis A₂ of the end effector 204 is substantially aligned with the longitudinal axis A₁ of the shaft 202, such that the end effector 204 is at a substantially zero angle relative to the shaft 202. Due to factors such as manufacturing tolerance and precision of measurement devices, the end effector 204 may not be at a precise zero angle relative to the shaft 202 in the unarticulated position, but nevertheless be considered “substantially aligned” thereto. In the articulated position, the longitudinal axes A₁, A₂ would be angularly offset from each other such that the end effector 204 is at a non-zero angle relative to the shaft 202.

In some embodiments, the surgical tool 200 may be supplied with electrical power (current) via a power cable 214 coupled to the housing 208. In other embodiments, the power cable 214 may be omitted and electrical power may be supplied to the surgical tool 200 via an internal power source, such as one or more batteries, capacitors, or fuel cells. In such embodiments, the surgical tool 200 may alternatively be characterized and otherwise referred to as an “electrosurgical instrument” capable of providing electrical energy to the end effector 204.

The power cable 214 may place the surgical tool 200 in electrical communication with a generator 216 that supplies energy, such as electrical energy (e.g., radio frequency energy), ultrasonic energy, microwave energy, heat energy, or any combination thereof, to the surgical tool 200 and, more particularly, to the end effector 204. Accordingly, the generator 216 may comprise a radio frequency (RF) source, an ultrasonic source, a direct current source, and/or any other suitable type of electrical energy source that may be activated independently or simultaneously.

In applications where the surgical tool 200 is configured for bipolar operation, the power cable 214 will include a supply conductor and a return conductor. Current can be supplied from the generator 216 to an active (or source) electrode located at the end effector 204 via the supply conductor, and current can flow back to the generator 216 via a return electrode located at the end effector 204 via the return conductor. In the case of a bipolar grasper with opposing jaws, for example, the jaws serve as the electrodes where the proximal end of the jaws are isolated from one another and the inner surface of the jaws (i.e., the area of the jaws that grasp tissue) apply the current in a controlled path through the tissue. In applications where the surgical tool 200 is configured for monopolar operation, the generator 216 transmits current through a supply conductor to an active electrode located at the end effector 204, and current is returned (dissipated) through a return electrode (e.g., a grounding pad) separately coupled to a patient's body.

The surgical tool 200 may further include a manual release switch 218 that may be manually actuated by a user (e.g., a surgeon) to open the jaws 210, 212. The release switch 218 is movably positioned on the drive housing 208, and a user is able to manually move (slide) the release switch 218 from a disengaged position, as shown, to an engaged position. In the disengaged position, the surgical tool 200 is able to operate as normal. As the release switch 218 moves to the engaged position, however, various internal component parts of the drive housing 208 are simultaneously moved, thereby resulting in the jaws 210, 212 opening, which might prove beneficial for a variety of reasons. In some applications, for example, the release switch 218 may be moved in the event of an electrical disruption that renders the surgical tool 200 inoperable. In such applications, the user would be able to manually open the jaws 210, 212 and thereby release any grasped tissue and remove the surgical tool 200. In other applications, the release switch 218 may be actuated (enabled) to open the jaws 210, 212 in preparation for cleaning and/or sterilization of the surgical tool 200. In some applications, the surgical tool 200 is first decoupled from the robotic manipulator and the associated motors, following which the user can actuate the manual release switch 218 to move the associated inputs and drive the cables once the motors are disengaged.

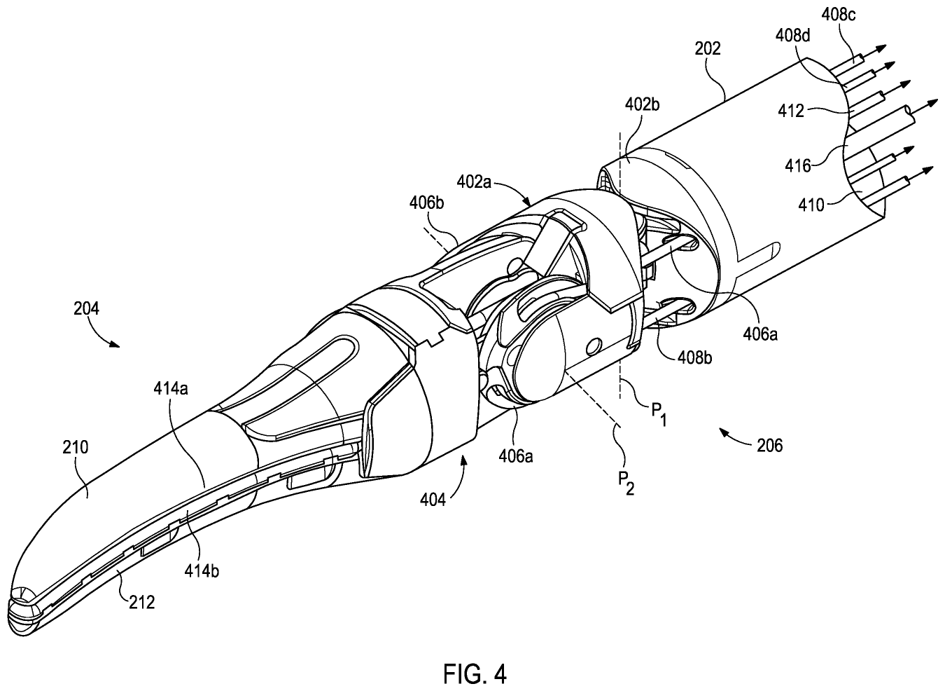

FIG. 4 is an enlarged isometric view of the distal end of the surgical tool 200. More specifically, FIG. 4 depicts an enlarged view of the end effector 204 and the wrist 206, with the jaws 210, 212 of the end effector 204 in the closed position. The wrist 206 operatively couples the end effector 204 to the shaft 202. In some embodiments, however, a shaft adapter may be directly coupled to the wrist 206 and otherwise interpose the shaft 202 and the wrist 206. Accordingly, the wrist 206 may be operatively coupled to the shaft 202 either through a direct coupling engagement where the wrist 206 is directly coupled to the distal end of the shaft 202, or an indirect coupling engagement where a shaft adapter interposes the wrist 206 and the distal end of the shaft 202. As used herein, the term “operatively couple” refers to a direct or indirect coupling engagement between two components.

To operatively couple the end effector 204 to the shaft 202, the wrist 206 includes a first or “distal” clevis 402a and a second or “proximal” clevis 402b. The clevises 402a,b are alternatively referred to as “articulation joints” of the wrist 206 and extend from the shaft 202, or alternatively a shaft adapter. The clevises 402a,b are operatively coupled to facilitate articulation of the wrist 206 relative to the shaft 202. As illustrated, the wrist 206 also includes a linkage 404 arranged distal to the distal clevis 402a and operatively mounted to the jaws 210, 212.

As illustrated, the proximal end of the distal clevis 402a may be rotatably mounted or pivotably coupled to the proximal clevis 402b at a first pivot axis P₁ of the wrist 206. In some embodiments, an axle may extend through the first pivot axis P₁ and the distal and proximal clevises 402a,b may be rotatably coupled via the axle. In other embodiments, however, such as is depicted in FIG. 4, the distal and proximal clevises 402a,b may be engaged in rolling contact, such as via an intermeshed gear relationship that allows the clevises 402a,b to rotate relative to each other similar to a rolling joint.

First and second pulleys 406a and 406b may be rotatably mounted to the distal end of the distal clevis 402a at a second pivot axis P₂ of the wrist 206. The linkage 404 may be arranged distal to the second pivot axis P₂ and operatively mounted to the jaws 210, 212. The first pivot axis P₁ is substantially perpendicular (orthogonal) to the longitudinal axis A₁ of the shaft 202, and the second pivot axis P₂ is substantially perpendicular (orthogonal) to both the longitudinal axis A₁ and the first pivot axis P₁. Movement of the end effector 204 about the first pivot axis P₁ provides “yaw” articulation of the wrist 206, and movement about the second pivot axis P₂ provides “pitch” articulation of the wrist 206.

A plurality of drive cables, shown as drive cables 408a, 408b, 408c, and 408d, extend longitudinally within a lumen 410 defined by the shaft 202 (or a shaft adaptor) and extend at least partially through the wrist 206. The drive cables 408a-d may form part of the cable driven motion system housed within the drive housing 208 (FIG. 2), and may comprise cables, bands, lines, cords, wires, woven wires, ropes, strings, twisted strings, elongate members, belts, shafts, flexible shafts, drive rods, or any combination thereof. The drive cables 408a-d can be made from a variety of materials including, but not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof. While four drive cables 408a-d are depicted in FIG. 4, more or less than four may be employed, without departing from the scope of the disclosure.

The drive cables 408a-d extend proximally from the end effector 204 and the wrist 206 toward the drive housing 208 (FIG. 2) where they are operatively coupled to various actuation mechanisms or devices that facilitate longitudinal movement (translation) of the drive cables 408a-d within the lumen 410. Selective actuation of the drive cables 408a-d applies tension (i.e., pull force) to the given drive cable 408a-d in the proximal direction, which urges the given drive cable 408a-d to translate longitudinally within the lumen 410.

In the illustrated embodiment, the drive cables 408a-d each extend longitudinally through the proximal clevis 402b. The distal end of each drive cable 408a-d terminates at the first or second pulleys 406a,b, thus operatively coupling each drive cable 408a-d to the end effector 204. In some embodiments, the distal ends of the first and second drive cables 408a,b may be coupled to each other and terminate at the first pulley 406a, and the distal ends of the third and fourth drive cables 408c,d may be coupled to each other and terminate at the second pulley 406b. In at least one embodiment, the distal ends of the first and second drive cables 408a,b and the distal ends of the third and fourth drive cables 408c,d may each be coupled together at corresponding ball crimps (not shown) mounted to the first and second pulleys 406a,b, respectively.

In at least one embodiment, the drive cables 408a-d may operate “antagonistically”. More specifically, when the first drive cable 408a is actuated (moved), the second drive cable 408b naturally follows as coupled to the first drive cable 408a, and when the third drive cable 408c is actuated, the fourth drive cable 408d naturally follows as coupled to the third drive cable 408c, and vice versa. Antagonistic operation of the drive cables 408a-d can open or close the jaws 210, 212 and can further cause the end effector 204 to articulate at the wrist 206. More specifically, selective actuation of the drive cables 408a-d in known configurations or coordination can cause the end effector 204 to articulate about one or both of the pivot axes P₁, P₂, thus facilitating articulation of the end effector 204 in both pitch and yaw directions. Moreover, selective actuation of the drive cables 408a-d in other known configurations or coordination will cause the jaws 210, 212 to open or close. Antagonistic operation of the drive cables 408a-d advantageously reduces the number of cables required to provide full wrist 206 motion, and also helps eliminate slack in the drive cables 408a-d, which results in more precise motion of the end effector 204.

In the illustrated embodiment, the end effector 204 is able to articulate (move) in pitch about the second or “pitch” pivot axis P₂, which is located near the distal end of the wrist 206. Thus, the jaws 210, 212 open and close in the direction of pitch. In other embodiments, however, the wrist 206 may alternatively be configured such that the second pivot axis P₂ facilitates yaw articulation of the jaws 210, 212, without departing from the scope of the disclosure.

In some embodiments, an electrical conductor 412 may also extend longitudinally within the lumen 410, through the wrist 206, and terminate at electrodes 414a, 414b to supply electrical energy to the end effector 204. In some embodiments, the electrical conductor 412 may comprise a wire, but may alternatively comprise a rigid or semi-rigid shaft, rod, or strip (ribbon) made of a conductive material. The electrical conductor 412 may be entirely or partially covered with an insulative covering (overmold) made of a non-conductive material. Using the electrical conductor 412 and the electrodes 414a,b, the end effector 204 may be configured for monopolar or bipolar RF operation.

In the illustrated embodiment, the end effector 204 comprises a combination tissue grasper and vessel sealer that includes a knife assembly (not visible), alternately referred to as a “cutting element” or “blade.” The knife is aligned with and configured to traverse a guide track or “knife slot” (not visible) defined longitudinally in one or both of the upper and lower jaws 210, 212. The knife may be operatively coupled to the distal end of a drive rod 416 (alternately referred to as “knife rod,” “actuation rod,” or “push rod”) that extends longitudinally within the lumen 410 and passes through the wrist 206. Longitudinal movement (translation) of the drive rod 416 correspondingly moves the knife within the knife slot(s). Similar to the drive cables 408a-d, the drive rod 416 may form part of the actuation systems housed within the drive housing 208 (FIG. 2). Selective actuation of a corresponding drive input will cause the drive rod 416 to move distally or proximally within the lumen 410, and correspondingly move the knife in the same longitudinal direction.

The drive rod 416 may comprise a rigid or semi rigid elongate member, such as a rod or shaft (e.g., a hypotube, a hollow rod, a solid rod, etc.), a wire, a ribbon, a push cable, or any combination thereof. The drive rod 416 can be made from a variety of materials including, but not limited to, metal (e.g., tungsten, nitinol, stainless steel, etc.), a polymer, or a composite material. The drive rod 416 may have a circular cross-section, but may alternatively exhibit a polygonal cross-section without departing from the scope of the disclosure.

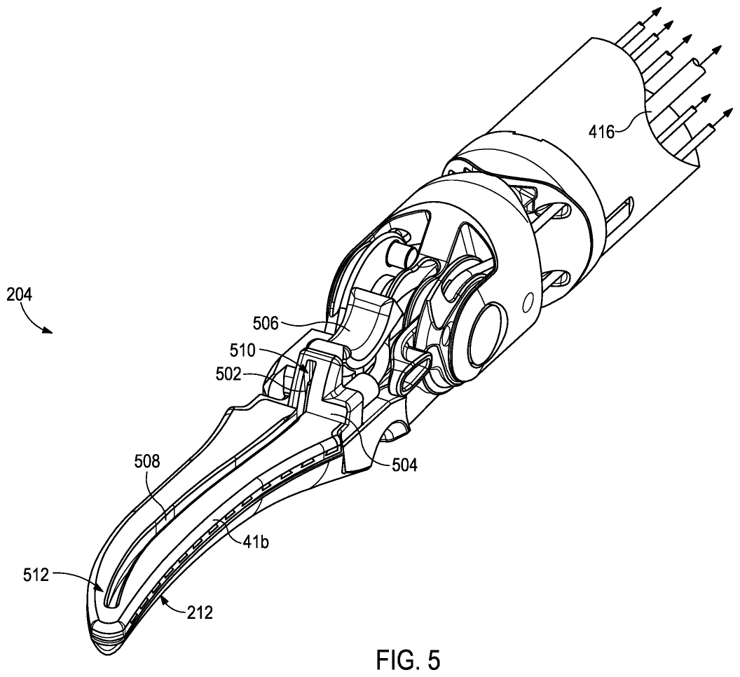

FIG. 5 is another enlarged isometric view of the end effector 204, according to one or more embodiments of the present disclosure. The upper jaw 210 (FIGS. 2 and 4) is omitted from FIG. 5 to enable viewing of various internal features of the end effector 204.

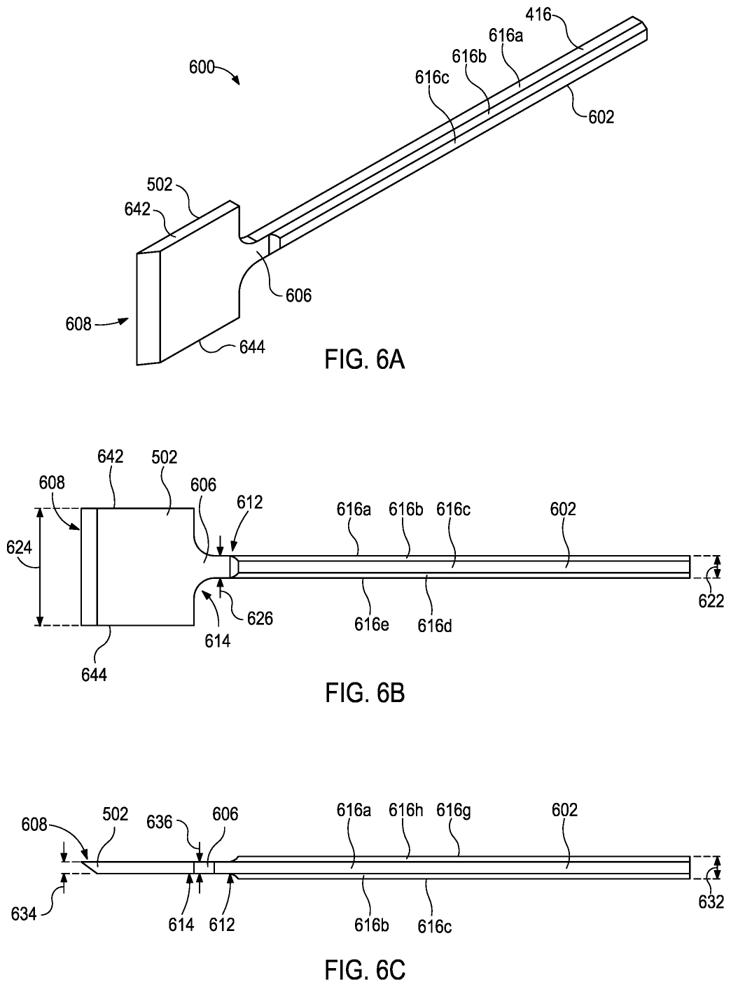

In the illustrated embodiment, a knife blade 502 (mostly occluded) of a knife assembly 600 (see FIGS. 6A-6C, below) is shown received within a portion of the electrode 414b of the lower jaw 212 and, more particularly, within a portion of an insulator 504 coupled to the electrode 414b. In its retracted position, as shown in FIG. 5, the knife blade 502 (hereinafter, the blade 502) may also be partially received within a knife housing 506 mounted to the end effector 204 between the upper and lower jaws 210, 212. The lower jaw 212 provides or otherwise defines a knife slot 508 through which the blade 502 may traverse upon distal actuation of the drive rod 416. While the knife slot 508 is shown provided in the lower jaw 212, in some embodiments, the knife slot 508 may be cooperatively defined by both the upper and lower jaws 210, 212. In embodiments, the knife slot 508 may be a substantially straight passageway; however, as shown in FIG. 5, the knife slot 508 may define a curved passageway.

As described in more detail below, the knife housing 506 defines a central passageway through which the drive rod 416 is able to extend to move the blade 502 along the knife slot 508. Upon firing the end effector 204, the drive rod 416 is moved (urged) distally, which correspondingly moves the blade 502 out of the knife housing 506 and into the knife slot 508. After firing is complete, the drive rod 416 is retracted proximally, which pulls the blade 502 proximally and back into the knife housing 506 until it is desired to again fire the end effector 204.

In FIG. 5, the blade 502 is shown in a first or “stowed” position, where the blade 502 is at least partially received within a cavity 510 defined by the insulator 504 and the knife housing 506. The cavity 510 is sized to receive and “stow” the blade 502 when not in use. When activated (or fired), the blade 502 is moved distally out of the cavity 510 into a second or “extended” position such that the blade 502 is operable to cut tissue. While not illustrated, when extended fully to the extended position, the blade 502 will be positioned at a distal end 512 of the knife slot 508 in either the upper or lower jaws 210, 212.

FIGS. 6A to 6C depict an example monolithic knife assembly 600 utilizable with the end effector 204, according to one or more embodiments. In particular, FIG. 6A depicts an isometric view of the monolithic knife assembly 600, FIG. 6B depicts a side view of the monolithic knife assembly 600, and FIG. 6C depicts a top view of the monolithic knife assembly 600.

The monolithic knife assembly 600 includes the blade 502 and the drive rod 416, such as a distal portion 602 of the drive rod 416. As illustrated, the blade 502 extends distally from the distal portion 602. As described above, the blade 502 is extendable through the knife slot 508 (FIG. 5). The distal portion 602 of the drive rod 416 is a length of the drive rod 416 proximate to a distal end thereof and, therefore, the distal portion 602 may simply be referred to herein as the “drive rod 602”. The drive rod 602 is thus operable in a similar manner as described above with respect to the drive rod 416, and therefore the drive rod 602 is similarly actuatable to extend (i.e., activate or fire) the blade 502.

Also, a cutting edge 608 is formed along a leading edge (at a distal end) of the blade 502. The cutting edge 608 of the blade 502 is the sharp portion of the blade 502 opposite a proximal or “trailing” edge 614 of the blade 502 and configured to sever or otherwise cut through tissue as the end effector 204 (FIGS. 2 and 4) fires and advances the blade 502 during operation. The cutting edge 608 may extend substantially orthogonal or perpendicular to a centerline or longitudinal axis of the drive rod 416.

In addition, the monolithic knife assembly 600 includes a hinge 606 interposing the blade 502 and the distal portion 602 of the drive rod 416. The hinge 606 represents the structural junction between the blade 502 and the distal portion 602 of the drive rod 416. As depicted, the hinge 606 extends from a distal end 612 of the distal portion 602 to a proximal edge 614 of the blade 502, wherein the proximal edge 614 is located opposite the cutting edge 608. Stated differently, the hinge 606 extends between the distal end 612 of the distal portion 602 and the proximal edge 614 of the blade 502.

The monolithic knife assembly 600 is made from a single piece of material. Thus, the drive rod 416 (including the distal portion 602), the blade 502, and the hinge 606 are all integral with each other and manufactured from a continuous, single piece of material, such as a single and continuous piece of nitinol. Accordingly, the drive rod 416 (including the distal portion 602), the blade 502, and the hinge 606 are all of the same type of material and manufactured from a single and continuous piece of material. In embodiments, the drive rod 416 (including the distal portion 602), the blade 502, and the hinge 606 are constructed from a single piece of nitinol, a single piece of stainless steel, or a single piece of Titanium.

The distal portion 602 may have various geometries that help provide a uniform bending modulus. In some embodiments, the remaining portion of the drive rod 416 extending proximally from the distal portion 602 may have the same geometry as the distal portion 602, but could alternatively exhibit a different geometry. In particular, the drive rod 416 (including the distal portion 602) may have a nearly circular geometry when evaluated in cross-section, as a circular geometry will provide the distal portion 602 with uniform (or near uniform) bending modulus in all directions. In embodiments, the distal portion 602 exhibits a polygonal shape in cross-section and, in the illustrated embodiment, the polygonal shape of the distal portion 602 is an octagon when evaluated in cross section.

As illustrated, the distal portion 602 having the octagonal shape includes eight (8) sidewalls 616a-616h; however, the distal portion 602 may have other polygonal shapes, with more or less than the eight (8) sidewalls 616a-616h, including without limitation, a dodecagon, a decagon, a hexagon, or a square, etc. In embodiments, the distal portion 602 of the drive rod 416 may have a different geometry than the remaining portion of the drive rod 416 extending proximally from the distal portion 602. For example, the distal portion 602 may have a polygonal geometry in cross-section, and the proximal portion extending therefrom may have a circular geometry. In other embodiments, the distal portion 602 and the portion of the drive rod 416 extending proximally therefrom may have the same geometry (e.g., the drive rod 416 may be polygonal shaped in cross section along its entire length).

As shown in FIG. 6B, the distal portion 602 exhibits a height 622, the blade 502 exhibits a height 624, and the hinge 606 exhibits a height 626. Here, the height 622 of the distal portion 602 is equal to the height 626 of the hinge 606, and the height 624 of the blade 502 is greater than the heights 622, 626 of the distal portion 602 and the hinge 606.

As shown in FIG. 6C, the distal portion 602 of the drive rod 416 exhibits a thickness 632, the blade 502 exhibits a thickness 634, and the hinge 606 exhibits a thickness 636. In the illustrated embodiment, the thickness 634 of the blade 502 is equal to the thickness 636 of the hinge 606, and the thickness 632 of the distal portion 602 is greater than the thicknesses 634, 636 of the blade 502 and the hinge 606 (i.e., the thicknesses 604, 606 are equal and less than the thickness 632 of the distal portion 602.) By providing the blade 502 and the hinge 606 with a relatively thinner (smaller) thickness 634, 636 as compared to the thickness 632 of the distal portion 602, it is easier for the blade 502 and the hinge 606 to bend as the blade 502 travels within the knife slot 508 (FIG. 5), which may be curved. Thus, blade 502 may more easily traverse the knife slot 508.

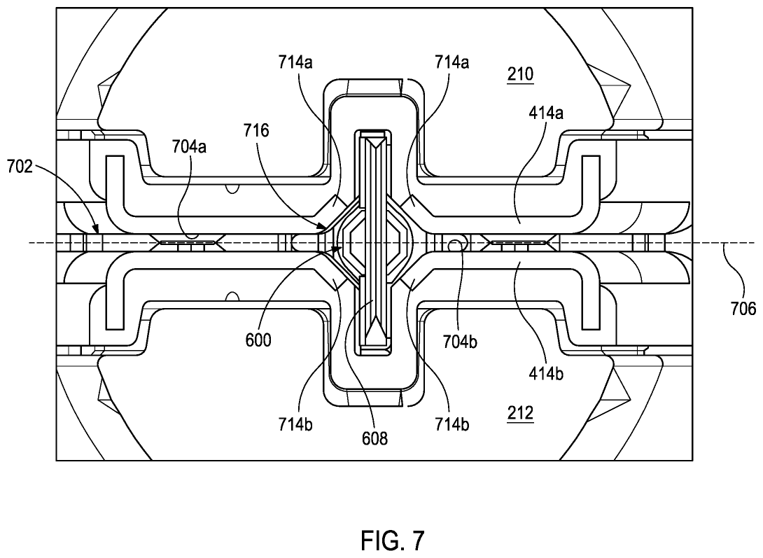

FIG. 7 depicts a cross-sectional end view of the end effector 204 depicting the monolithic knife assembly 600 arranged between the upper and lower jaws 210, 212 when in the closed position, according to one or more embodiments. As illustrated the end effector 204 includes the upper jaw 210 and the lower jaw 212, and a gap 702 is defined between the upper and lower jaws 210, 212 when the jaws 210, 212 are closed. The upper jaw 210 provides an upper surface 704a and the lower jaw 212 provides an opposing lower surface 704b. The upper surface 704a may be the same structure as the upper electrode 414a attached to or forming part of the upper jaw 210, and the lower surface 704b may be the same structure as the electrode 414b attached to or forming part of the lower jaw 212. In some embodiments, the upper surface 704a and/or the lower surface 704b may also comprise an electrode, but may otherwise comprise a planar sealing surface made of a nonconductive material (e.g., an insulator).

When the jaws 210, 212 are in the closed position, as shown in FIG. 7, the upper and lower surfaces 704a, 704b oppose each other and cooperatively form a sealing plane 706 that extends through the gap 702. The sealing plane 706 is alternately referred to as a “tissue capturing section” or “tissue plane” since the sealing plane 706 is the location where tissue can be grasped between the upper and lower jaws 210, 212 in preparation for cutting with the blade 502. As the blade 502 traverses the knife slot 508 (FIG. 5), the cutting edge 608 extends substantially perpendicular to the sealing plane 706.

In some embodiments, as illustrated, the jaws 210, 212 and electrodes 414a, 414b of the end effector 204 are arranged such that the sealing plane 706 bisects the cutting edge 608. In other embodiments, however, it is contemplated herein that the blade 502 may be differently arranged such that more or less of the cutting edge 608 is positioned above or below the sealing plane 706, without departing from the scope of the disclosure. Regardless, the cutting edge 608 may be arranged and otherwise configured to cut tissue that may be located below the sealing plane 706 (such as tissue that may migrate into the knife slot 508 in the lower jaw 212) and/or to cut tissue that may be located above the sealing plane 706 (such as tissue that may migrate into the knife slot 508 formed in the upper jaw 210) upon closing the jaws 210, 212.

Here, the drive rod 416 extends along (and coplanar with) the sealing plane 706. To provide clearance for the drive rod 416 that is centrally located in this manner, chamfers or angled surfaces are formed in the electrodes 414a, 414b at the knife slot 508. As shown, chamfers 714a are formed in the upper electrode 414a and chamfers 714b are formed in the lower electrode 414b, and the chamfers 714a, 714b together define a central channel or clearance 716 through which the drive rod 416 (and the distal portion 604 thereof) may extend and travel.

However, utilizing this type of centrally extending drive rod 416 architecture in combination with symmetric bifurcating jaw architecture having dual bi-polar electrodes presents challenges. The sealing ability of the electrodes 414a,b depends on the surface area of the upper and lower surfaces 704a,b, and the presence of the chamfers 714a,b effectively reduces the surface areas of the upper and lower surfaces 704a,b, which in turn adversely impacts sealing performance of the end effector 204. Thus, the sealing performance of the electrodes 414a,b may be maximized by minimizing the extent of the chamfers 714a,b. However, minimizing the chamfers 714a,b will correspondingly reduce the size of the central clearance 716, which needs to be appropriately sized to operably receive the drive rod 416 without abrasion. Accordingly, it is advantageous to reduce the cross-sectional size of size of the drive rod 416, such that the cross-sectional size of the central clearance 716 may be minimized to thereby correspondingly maximize the surface areas of the upper and lower surfaces 704a,b, which in turn results in improved sealing performance.

The monolithic knife assembly 600 described herein, which is formed of a homogeneous and single piece of material, incorporates the distal portion 602 of the drive rod 416 having a reduced cross-sectional area and being integrally attached to the blade 502, as compared to conventional knife assemblies that comprise separate components (such as a ferrule) to fasten the blade to the drive rod which result in a relatively larger cross-sectional area and which require a larger central clearance 716 through which it may translate. Further, the monolithic knife assembly 600 detailed herein, while being made from a homogeneous and single piece of material, is able to provide satisfactory tissue cutting properties and maintain adequate stiffness of the drive rod 416 to prevent buckling, while also providing sufficient bending modulus and yield strength to enable bending when maneuvered around the knife slot 508 and when articulated/bent by the wrist 206.

Various processes and techniques may be utilized to manufacture the monolithic knife assembly 600. For example, a photo chemical machining (“PCM”) process may be utilized to produce the monolithic knife assembly 600, and process parameter tuning and selective masking allows for the formation of the desired cross sectional shape of the distal portion 602 of the drive rod 416, as well as the general shape of the blade 604 and the leading cutting edge 608 that extends perpendicular to the top and bottom edges 642, 644 of the blade 604. In such examples, the manufacturing process may begin with a sheet of material (e.g., a Nitinol sheet, a stainless steel sheet, a Titanium sheet, or other flexible materials, etc.) and, through a series of PCM steps, the final geometry of the monolithic knife assembly 600 may be achieved.

Alternatively, the single piece of material (e.g., nitinol) may be machined (e.g., computer numerical control (“CNC”) machining) to form the monolithic knife assembly 600, with the blade 604 thereof being chemically etched, machined, or ground at an end of the single piece of material (e.g., the blade 604 may be etched or ground into a distal end of the single piece of nitinol). Alternatively, a direct laser metal sintering process may be utilized, where the single piece of material comprises nitinol, and then a secondary process (e.g., PCM) may be used to generate the blade 604 with sufficient sharpness, hardness, and durability. Alternatively, a metal injection molding process may be utilized. A metal injection molding process may be utilized to form the monolithic knife assembly 600, with the blade 604 thereof being chemically etched, machined, or ground, and materials utilized in such process may include Nitinol, stainless steel, Titanium, etc. Further, an additive manufacturing process may be utilized, where the single piece of material utilized is a liquid metal. An additive manufacturing process (i.e., a 3D printing process) may be used to form the monolithic knife assembly 600, with the blade 604 thereof being chemically etched, machined, or ground, and materials utilized in such process may include Nitinol, stainless steel, Titanium, etc.

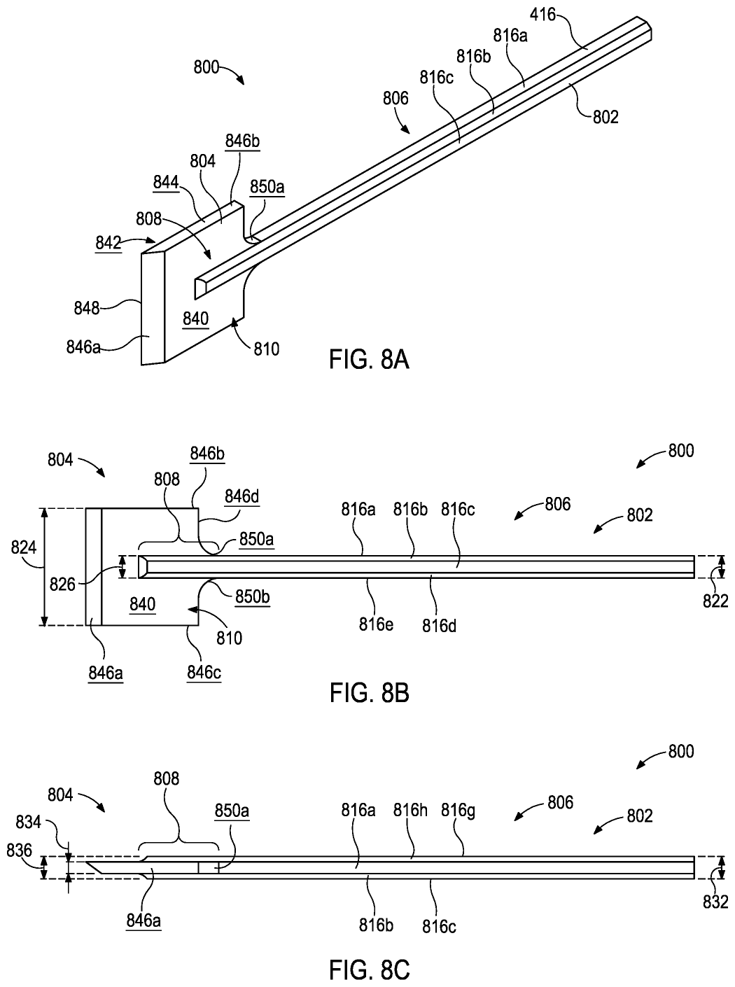

FIGS. 8A to 8C depict another example of a monolithic knife assembly 800 utilizable with the end effector 204, according to one or more additional embodiments. In particular, FIG. 8A depicts an isometric view of the monolithic knife assembly 800, FIG. 8B depicts a side view of the monolithic knife assembly 800, and FIG. 8C depicts a top view of the monolithic knife assembly 800. As with the monolithic knife assembly 600 detailed above, the monolithic knife assembly 800 includes a drive rod 802 and a knife blade 804 distally extending from the drive rod 802. The drive rod 802 is substantially similar to the drive rod 416 described above, and FIGS. 8A-8C depict a distal portion 806 of the drive rod 802, such that the skilled person will recognize that the drive rod 802 also includes a proximal portion (not illustrated) in FIGS. 8A-8C that extends proximally as detailed above with reference to the drive rod 416.

The knife blade 804 (hereinafter, the “blade 804”) is extendable through a knife slot, such as the knife slot 508 of FIG. 5. Further, the drive rod 802 (including the distal portion 806 thereof) and the blade 804 are made from a homogeneous piece of material (i.e., the same piece of material). Suitable materials from which the monolithic knife assembly 800 may be fabricated are mentioned above with reference to the monolithic knife assembly 600. Moreover, processes and techniques utilized to manufacture the monolithic knife assembly 800 are also described above with reference to the monolithic knife assembly 600 and therefore will not be discussed again in detail.

In the illustrated example, the drive rod 802 includes a distal extension 808 that extends from the distal end 806 and overlaps a portion of the blade 804, such that the blade 804 includes the distal extension 808 and a remaining portion 810 that is not overlapped by the drive rod 802.

As shown in FIG. 8B, the distal portion 806 exhibits a height 822 and the blade 804 exhibits a height 824. Also, the distal extension 808 has a height 826. As shown in FIG. 8C, the distal portion 806 of the drive rod 802 exhibits a thickness 832 and the blade 804 exhibits a thickness 834. Also shown in FIG. 8C, the distal extension 808 of the blade 804 exhibits a thickness 836.

With this overlapping arrangement of the drive rod 802 and the blade 804, the thickness 836 of the distal extension 808 is equal to the thickness 832 of the drive rod 802 (i.e., thicknesses 832, 836 are equal), and the thicknesses 832, 836 are larger (greater) than the thickness 834 of the remaining portion 810 of the blade 804 that is not overlapped by the drive rod 802. Also, in the illustrated example, the height 826 of the distal extension 808 is equal to the height 822 of the drive rod 802 (i.e., heights 822, 826 are equal), and the height 824 of the blade 804 is larger (greater) than the heights 822, 826 of the drive rod 802 and the distal extension 808 (i.e., the heights 822, 826 are equal and lesser than the height 824). In other embodiments, the height 824 of the blade 804 may be equal to either or both of the heights 822, 826, or the height 824 of the blade 804 may be less than either or both of the heights 822, 826.

As with the monolithic knife assembly 600 of FIGS. 6A-6C, the distal portion 806 may have various geometries that help provide a uniform bending modulus and, in embodiments, the remaining portion of the drive rod 802 extending proximally from the distal portion may have the same geometry as the distal portion 806. In particular, the drive rod 802 (including the distal portion 806) may have a nearly circular cross-sectional geometry, as such geometry will help provide uniform bending modulus in all directions. In the illustrated embodiment, the distal portion 806 exhibits a polygonal cross-sectional shape and, in particular, an octagonal cross section. With an octagonal cross-section, the distal portion 806 includes eight (8) sidewalls 816a-816h. However, the distal portion 806 may alternatively exhibit other polygonal cross-sections with more or less than the eight (8) sidewalls 816a-816h, including without limitation, a dodecagon, a decagon, a hexagon, or a square, etc.

In some embodiments, the distal portion 806 of the drive rod 802 may have a different geometry than the remaining portion of the drive rod 802 extending proximally from the distal portion 806. For example, the distal portion 806 may exhibit a polygonal cross-section, and the proximal portion extending therefrom may exhibit a circular cross-section. In other embodiments, the distal portion 806 and the portion of the drive rod 802 extending proximally therefrom may have the same geometry (e.g., the drive rod 802 may exhibit a polygonal cross-section along its entire length).

The blade 804 includes a first face 840, a second face 842 that is opposite the first face 840, and a peripheral sidewall 844 extending between the first face 840 and the second face 842. The peripheral sidewall 844 includes a distal sidewall 846a, a top sidewall 846b, a bottom sidewall 846c opposite the bottom sidewall 846b, and a proximal sidewall 846d opposite the distal sidewall 846a. The distal sidewall 846a is obliquely oriented relative to the first face 840 and the second face 842, such that a leading cutting edge 848 is defined at the junction between the distal sidewall 846a and the second face 842. Also, in the illustrated embodiment, the proximal sidewall 846d includes upper and lower curved portions 850a, 850b at which the proximal sidewall 846d blends or transitions into the drive rod 802.

As mentioned above, FIGS. 8A-8C depict an embodiment where the monolithic knife assembly 800 includes the distal extension 808 overlapping and extending over a portion of the blade 804. With this geometry, and as shown in at least FIG. 8B, the sidewalls 816b, 816c, 816d extend over the first face 840 of the blade 804 and the sidewalls 816f, 816g, 816h extend over the second face 842 of the blade 804. Also in the illustrated embodiment, the sidewalls 816a and 816e extend into the top and bottom sidewalls 846b, 846c, respectively at the upper and lower curved portions 850a, 850b. The upper and lower curved portions 850a, 850b are operable to help reduce stresses at the junction to the blade 804 due to flexural tensile and compressive loads.

Embodiments disclosed herein include:
A. An end effector for a surgical tool include opposing first and second jaws, and a knife slot defined in one or both of the first and second jaws, and a monolithic knife assembly comprising a drive rod and a blade distally extending from the drive rod and extendable through the knife slot, the drive rod and the blade being made from a same material.
B. An end effector for a surgical tool includes opposing first and second jaws, and a knife slot defined in one or both of the first and second jaws, and a monolithic knife assembly comprising a drive rod exhibiting a polygonal cross-sectional shape, a hinge integrally extending from a distal end of the drive rod, and a blade distally extending from the hinge and being extendable through the knife slot, wherein the drive rod, the hinge, and the blade are integrally formed and made from a same material.
C. A method of operating a surgical tool includes positioning the surgical tool adjacent a patient for operation, the surgical tool including a drive housing, an elongate shaft extending from the drive housing, a drive rod extending from the drive housing within the elongate shaft, and an end effector arranged at a distal end of the elongate shaft, the end effector including opposing first and second jaws, a knife slot defined in one or both of the first and second jaws, and a blade integrally extending from a distal end of the drive rod and extendable through the knife slot, the drive rod and the blade being made from a same material. The method further includes closing the opposing first and second jaws and thereby grasping onto tissue between the opposing first and second jaws, and actuating the drive rod to advance the blade through the knife slot and cut the tissue.

Each of embodiments A, B, and C may have one or more of the following additional elements in any combination: Element 1: further comprising a hinge interposing the blade and the drive rod, wherein the hinge is integrally formed with the blade and the drive rod such that the hinge is made from the same material. Element 2: wherein the hinge and the drive rod exhibit an equal height that is less than a height of the blade, and wherein the hinge and the blade exhibit an equal thickness. Element 3: wherein the equal thickness of the hinge and the blade is less than a thickness of the drive rod. Element 4: further comprising a distal extension that extends from a distal end of the drive rod and overlaps a portion of the blade. Element 5: wherein the drive rod and the distal extension exhibit an equal thickness that is larger than a thickness exhibited by a remaining portion of the blade not overlapped by the distal extension. Element 6: wherein the drive rod and the distal extension exhibit an equal height that is less than a height of the blade. Element 7: wherein a leading edge of the blade includes a cutting edge extending perpendicular to a sealing plane provided between the opposing first and second jaws when the opposing first and second jaws are closed. Element 8: wherein the drive rod exhibits a polygonal cross-sectional shape. Element 9: wherein the polygonal cross-sectional shape is selected from the group consisting of an octagon, a hexagon, and a square. Element 10: wherein the same material comprises nitinol, stainless steel, or titanium. Element 11: wherein the monolithic knife assembly is manufactured via a process selected from the group consisting of photo chemical machining, machining where the same material comprises stainless steel or nitinol, direct laser metal sintering where the same material comprises nitinol, metal injection molding, additive manufacturing where the same material comprises a liquid metal.

Element 12: wherein the hinge and the drive rod exhibit an equal height that is less than a height of the blade, and wherein the hinge and the blade exhibit an equal thickness. Element 13: wherein the equal thickness of the hinge and the blade is less than a thickness of the drive rod. Element 14: further comprising a distal extension that extends from a distal end of the drive rod and overlaps a portion of the blade. Element 15: wherein the polygonal cross-sectional shape is octagonal.

Element 16: wherein a leading edge of the blade includes a leading cutting edge extending perpendicular to a sealing plane provided between the opposing first and second jaws when the opposing first and second jaws are closed, the method further comprising cutting tissue grasped between the opposing first and second jaws with the cutting edge as the blade advances through the knife slot. Element 17: wherein the drive rod exhibits a polygonal cross-sectional shape.

By way of non-limiting example, exemplary combinations applicable to A, B, and C include: Element 1 with Element 2; Element 2 with Element 3; Element 4 with Element 5; Element 4 with Element 6; Element 8 with Element 9; and Element 12 with Element 13.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. An end effector for a surgical tool, comprising:
opposing first and second jaws, and a knife slot defined in one or both of the first and second jaws; and
a monolithic knife assembly comprising a drive rod and a blade distally extending from the drive rod and extendable through the knife slot, the drive rod and the blade being made from a same material.

2. The end effector of claim 1, further comprising a hinge interposing the blade and the drive rod, wherein the hinge is integrally formed with the blade and the drive rod such that the hinge is made from the same material.

3. The end effector of claim 2, wherein the hinge and the drive rod exhibit an equal height that is less than a height of the blade, and wherein the hinge and the blade exhibit an equal thickness.

4. The end effector of claim 3, wherein the equal thickness of the hinge and the blade is less than a thickness of the drive rod.

5. The end effector of claim 1, further comprising a distal extension that extends from a distal end of the drive rod and overlaps a portion of the blade.

6. The end effector of claim 5, wherein the drive rod and the distal extension exhibit an equal thickness that is larger than a thickness exhibited by a remaining portion of the blade not overlapped by the distal extension.

7. The end effector of claim 5, wherein the drive rod and the distal extension exhibit an equal height that is less than a height of the blade.

8. The end effector of claim 1, wherein a leading edge of the blade includes a cutting edge extending perpendicular to a sealing plane provided between the opposing first and second jaws when the opposing first and second jaws are closed.

9. The end effector of claim 1, wherein the drive rod exhibits a polygonal cross-sectional shape.

10. The end effector of claim 9, wherein the polygonal cross-sectional shape is selected from the group consisting of an octagon, a hexagon, and a square.

11. The end effector of claim 1, wherein the same material comprises nitinol, stainless steel, or titanium.

12. The end effector of claim 1, wherein the monolithic knife assembly is manufactured via a process selected from the group consisting of photo chemical machining, machining where the same material comprises stainless steel or nitinol, direct laser metal sintering where the same material comprises nitinol, metal injection molding, additive manufacturing where the same material comprises a liquid metal.

13. An end effector for a surgical tool, comprising:
opposing first and second jaws, and a knife slot defined in one or both of the first and second jaws; and
a monolithic knife assembly comprising:
a drive rod exhibiting a polygonal cross-sectional shape;
a hinge integrally extending from a distal end of the drive rod; and
a blade distally extending from the hinge and being extendable through the knife slot,
wherein the drive rod, the hinge, and the blade are integrally formed and made from a same material.

14. The end effector of claim 13, wherein the hinge and the drive rod exhibit an equal height that is less than a height of the blade, and wherein the hinge and the blade exhibit an equal thickness.

15. The end effector of claim 14, wherein the equal thickness of the hinge and the blade is less than a thickness of the drive rod.

16. The end effector of claim 13, further comprising a distal extension that extends from a distal end of the drive rod and overlaps a portion of the blade.

17. The end effector of claim 13, wherein the polygonal cross-sectional shape is octagonal.

18. A method of operating a surgical tool, comprising:
positioning the surgical tool adjacent a patient for operation, the surgical tool including a drive housing, an elongate shaft extending from the drive housing, a drive rod extending from the drive housing within the elongate shaft, and an end effector arranged at a distal end of the elongate shaft, the end effector including:
opposing first and second jaws;
a knife slot defined in one or both of the first and second jaws; and
a blade integrally extending from a distal end of the drive rod and extendable through the knife slot, the drive rod and the blade being made from a same material,
closing the opposing first and second jaws and thereby grasping onto tissue between the opposing first and second jaws; and
actuating the drive rod to advance the blade through the knife slot and cut the tissue.

19. The method of claim 18, wherein a leading edge of the blade includes a leading cutting edge extending perpendicular to a sealing plane provided between the opposing first and second jaws when the opposing first and second jaws are closed, the method further comprising:
cutting tissue grasped between the opposing first and second jaws with the cutting edge as the blade advances through the knife slot.

20. The method of claim 18, wherein the drive rod exhibits a polygonal cross-sectional shape.
